(19) United States
(12) Patent Application Publication
(10) Pub. No.: US 2022/0184463 A1
(43) Pub. Date: Jun. 16, 2022

(54) SPORTS BALL SYSTEM FOR MONITORING BALL CHARACTERISTICS AND METHOD THEREFOR

(71) Applicant: MICHAEL GANSON, LAS VEGAS, NV (US)

(72) Inventor: MICHAEL GANSON, LAS VEGAS, NV (US)

(21) Appl. No.: 17/117,380

(22) Filed: Dec. 10, 2020

(51) Int. Cl. A63B 43/00; A63B 71/06; A63B 37/02; A63B 69/00; A63B 24/00

(52) U.S. Cl. CPC A63B 43/004; A63B 2220/833; A63B 37/02; A63B 69/0002; A63B 24/0021; A63B 2220/836; A63B 2071/0666; A63B 2071/0663; A63B 2071/0694; A63B 2220/40; A63B 2220/35; A63B 2225/50; A63B 2069/0006; A63B 2024/0034; A63B 71/0622

(57) ABSTRACT

A sports ball system for calculating movement characteristics has a sports ball. The sports ball monitors movement characteristics of the sports ball. A plurality of wearable devices is worn by a batter whom the sports ball is being thrown towards. At least one of the plurality of wearable devices receiving and displaying data transmitted by the sports ball and a remaining plurality of wearable devices recording data relating to movement of the batter.

### RELATED APPLICATIONS

The present application is a related to U.S. Pat. No. 9,522,306 entitled, “SPORTS BALL THAT MEASURES SPEED, SPIN, CURVE, MOVEMENT AND OTHER CHARACTERISTICS AND METHOD THEREFOR”, issued on Dec. 20,2016; U.S. Pat. No. 9,526,951 entitled “SPORTS BALL SYSTEM FOR MONITORING BALL CHARACTERISTICS AND METHOD THEREFOR”, issued Dec. 27, 2016 and patent application entitled, “SPORTS BALL SYSTEM FOR MONITORING BALL CHARACTERISTICS AND METHOD THEREFOR, filed Feb. 19, 2019, and having Ser. No. 16/279,832 all in the name of the same inventor as the present application and all being incorporated herewith.

### TECHNICAL FIELD

The present application generally relates to a sports ball, and more specifically to a balanced baseball that has the characteristic of a regulation baseball and measures other movement characteristics as deemed relevant to include the speed, spin rate, curve of the baseball.

### BACKGROUND

In many sports it is desired to determine how fast a ball is being either thrown or hit. Typically, the speed of a moving ball is measured using a Doppler radar system. Doppler radar systems determine a moving ball's speed by analyzing radar beams reflected off the ball. Although accurate, these systems are expensive and normally cannot be operated by the athlete whose toss or hit is being measured. For these reasons, systems of this type are generally restricted to organized sport teams.

Just as important to speed is to know the motion characteristics of the ball, such as the distance, time of flight, speed, height, spin rate, curve, release point or other motion characteristics of the thrown or batted ball. All of the above characteristics may be used to help a pitcher optimize different types of pitches. For example, spin rate information is useful for example in optimizing a baseball pitcher's curve ball pitching ability. Unfortunately, the above motion characteristics are difficult to measure. In general, the above motion characteristics are generally calculated by videotaping a pitch and having a complex computer program analyze the different motion characteristics. Thus, as with speed, systems of this type are generally restricted to organized sport teams.

Thus, the ability to measure and review different motion characteristics of a pitched ball is generally reserved for professional sporting teams. The typical amateur ball player is unable to measure, review and analyze the different motion characteristics of a pitched ball.

Presently, there are sports balls which allow one to track the speed and other characteristics of the thrown and/or hit ball. However, these balls are not regulation caliber. Thus, the correlation between readings from these sports balls and throwing a regulation sports ball may not be accurate. Further, many of these sports ball are not balanced. Throwing an unbalanced sports ball creates false motion characteristics and could result in throwing injuries.

Therefore, it would be desirable to provide a system and method that overcomes the above.

### SUMMARY

In accordance with one embodiment, a sports ball system for calculating movement characteristics is disclosed. The sports ball system uses a sports ball for monitoring and calculating the movement characteristics of the sports ball. A plurality of wearable devices is worn by a batter whom the sports ball is being thrown towards. At least one of the plurality of wearable devices receives and displays data transmitted by the sports ball and a remaining plurality of wearable devices recording data relating to movement of the batter.

In accordance with one embodiment, a sports ball system for calculating movement characteristics is disclosed. The sports ball system has a sports ball. The sports ball records and transmits movement characteristics of the sports ball. A plurality of wearable devices worn by a batter whom the sports ball is being thrown towards are provided. At least one of the plurality of wearable devices receives and displays data transmitted by the sports ball. A remaining plurality of wearable devices record data relating to movement of the batter. The at least one of the plurality of wearable devices receiving and displaying data transmitted by the sports ball is a pair of smart glasses worn by the batter.

In accordance with one embodiment, a sports ball system for calculating movement characteristics is disclosed. The sports ball system has a sports ball. The sports ball records and transmits movement characteristics of the sports ball. A plurality of wearable devices is worn by a batter whom the sports ball is being thrown towards. At least one of the plurality of wearable devices is a pair of smart glasses worn by the batter receiving and displaying data transmitted by the sports ball. A remaining plurality of wearable devices records data relating to movement of the batter. The remaining plurality of wearable devices comprises: at least one wrist sensor; at least one foot sensor; at least one waist sensor; and at least one batting helmet sensor.

### BRIEF DESCRIPTION OF THE DRAWINGS

The present application is further detailed with respect to the following drawings. These figures are not intended to limit the scope of the present application but rather illustrate certain attributes thereof. The same reference numbers will be used throughout the drawings to refer to the same or like parts.

FIG. 1 is a prospective view of a sports ball for measuring a plurality of motion characteristics in accordance with one aspect of the present application;

FIG. 2A-2D are prospective view of the different components of the sports ball of FIG. 1 in accordance with one aspect of the present application;

FIG. 3 is a cutaway view of the sports ball of FIG. 1 in accordance with one aspect of the present application;

FIG. 4 is a magnified view of the measuring electronics and mounting system of the sports ball of FIG. 1 in accordance with one aspect of the present application;

FIG. 5 is another magnified view of the measuring electronics and mounting system of the sports ball of FIG. 1 in accordance with one aspect of the present application;

FIG. 6 is a front view of a wearable device used with sports ball of FIG. 1 in accordance with one aspect of the present application;

FIG. 7 is a front view of an individual having a plurality of wearable devices used with the sports ball of FIG. 1 in accordance with one aspect of the present application;

FIG. 8A-8G is a side view of the individual in FIG. 7 having the plurality of wearable devices used with the sports ball of FIG. 1 in accordance with one aspect of the present application;

FIG. 9 is a block diagram of the wearable device used with the sports ball of FIG. 1 in accordance with one aspect of the present application;

FIG. 10 is a front view of a monitoring device with a shield used with the sports ball of FIG. 1 in accordance with one aspect of the present application;

FIG. 11 is a front view of a monitoring device with the shield removed used with the sports ball of FIG. 1 in accordance with one aspect of the present application;

FIG. 12 is a side view of a monitoring device with the shield removed used with the sports ball of FIG. 1 in accordance with one aspect of the present application;

FIG. 13 is a block diagram of a monitoring unit used with the monitoring device of FIG. 10 in accordance with one aspect of the present application;

FIG. 14 is a front view of a wearable device used with sports ball of FIG. 1 in accordance with one aspect of the present application; and

FIG. 15 is a block diagram of a system using the sports ball of FIG. 1 in accordance with one aspect of the present application.

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure

Embodiments of the exemplary system and method disclose a regulation sports ball that is able to measure and record motion characteristics of the sports ball when the sports ball is thrown. The sports ball may be able to measure and record the speed, distance, time of flight, height, path, spin rate, curve, release point or other motion characteristics of the thrown ball. All of the above characteristics may be used to help a pitcher optimize different types of pitches.

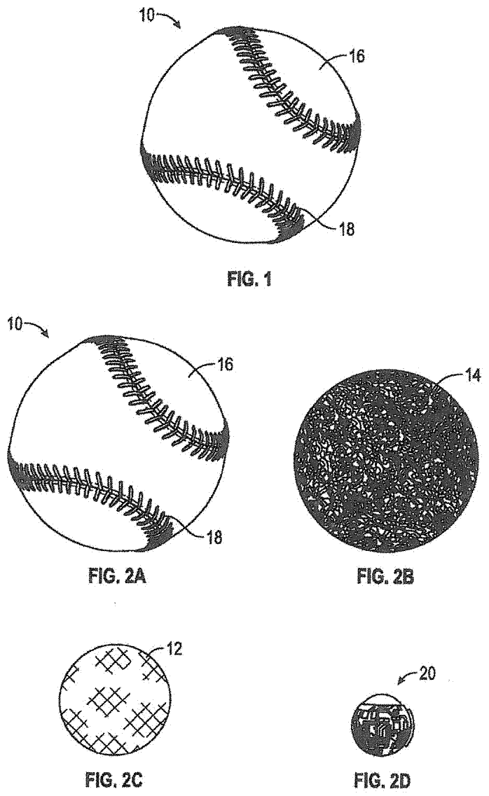

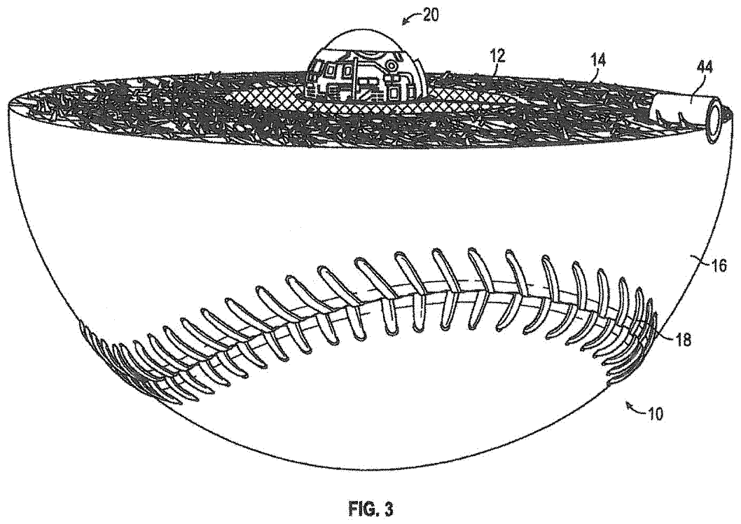

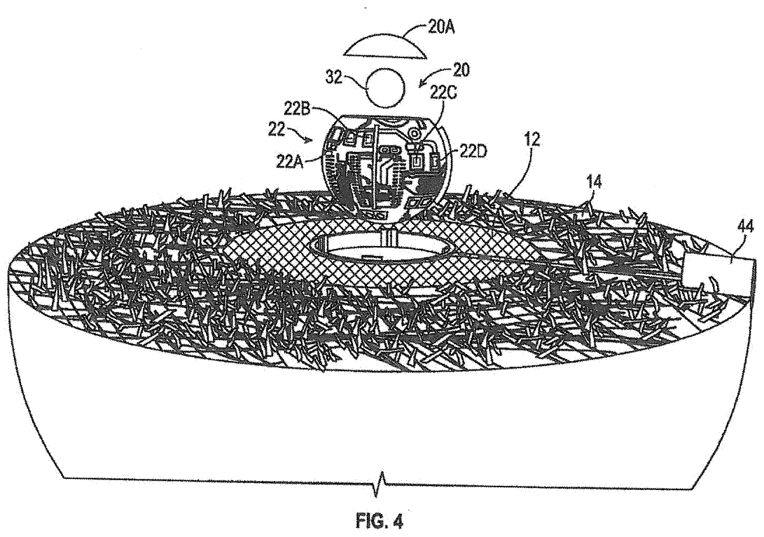

Referring to the FIGS. 1-5, a sports ball 10 is shown. The sports ball 10 may be constructed to measure and record the speed, distance, time of flight, height, path, spin rate, curve, release point or other motion characteristics of the thrown sports ball 10. The sports ball 10 may be configured to conform to a regulation ball such as a baseball, bowling ball or other regulation sports balls. While embodiments shown in the Figures disclose a baseball, this should not be seen in a limiting manner.

In the embodiment shown, the sports ball 10 is a regulation baseball. Thus, the sports ball 10 may be configured to be between 9 and 9¼ inches in circumference, and 5 to 5¼ ounces in weight, and have a coefficient of restitution of no more than 0.578, no less than 0.514.

The sports ball 10 may be constructed in a similar manner to a regulation baseball. The sports ball 10 may have a spherical core 12. The spherical core 12 may be formed of cork, rubber or similar material. A winding 14 may be formed around the spherical core 12. The winding 14 may be formed of wool, poly/cotton or other winding material. A covering 16 may be formed around the winding 14 encompassing the core 12 forming an exterior surface of the sports ball 10. The covering 16 may be formed out of cowhide or similar material. The covering 16 may be formed out of natural or synthetic materials. The covering 16 may be formed in two hourglass shaped halves which may be couple together around the winding 14 encompassing the core 12 by stitching 18.

The sports ball 10 has a spherical circuit board 20. The spherical circuit board 20 may have a hollow interior section. Alternatively, the spherical circuit board 20 may be solid wherein the interior of the spherical circuit board 20 is a non-conductive substrate. A plurality of sensors 22 may be coupled to the spherical circuit board 20. The sensors 22 may be used to monitor different motion characteristics of the thrown sports ball 10. The sensors 22 may include: an accelerometer 22A to measure the speed of the thrown sport ball 10; gyroscope 22B to measure the rotation of the thrown sports ball 10; a position sensor 22C to monitor a location of the thrown sport ball 10; and a pressure sensor 22D to measure barometric pressure as to quantify a change in altitude as the sports ball 10 changes in elevation during delivery and to quantify atmospheric air density in the sports ball 10. The above list is given as an example. Other sensors may be used to measure different motion characteristics without departing from the spirit and scope of the present invention. In accordance with one embodiment, the sports ball 10 may be customized. Thus, a potential buyer of the sports ball 10 may designate which sensors 22 the buyer wishes to incorporate into the sports ball 10.

In accordance with one embodiment, the accelerometer 22A may be a package containing both an accelerometer and a magnetometer. For example, the accelerometer 22A may be an LSM303AH accelerometer made by STMicorelectronics®. The LSM303AH accelerometer 22A is an ultra-low-power high performance system-in-package featuring a 3D digital linear acceleration sensor 22A′ and a 3D digital magnetic sensor 22A″. The 3D digital linear acceleration sensor 22A′ may contain three separate accelerometers oriented in three orthogonal axes. A useful internal feature calculates the vector magnitude of those three axes. The accelerometer 22A may communicate with a processor 24 disclosed below either over a 2-wire I2C interface or through a faster 4-wire SPI interface. Due to the need for fast response times, the 4-wire SPI interface may be used.

The 3D digital linear acceleration sensor 22A′ may include several control signals that can be programmed to generate an interrupt signal once a certain threshold condition is met. This may allow the sports ball 10 to be programmed to place the sports ball 10 into a very low-power sleep state to save battery power. When the 3D digital linear acceleration sensor 22A′ monitors forces/sequence that may exceed a preset level, the accelerometer may generate an interrupt signal that will in turn cause the sports ball 10 to wake-up. A programmed algorithm may determine if this is the correct wake-up sequence, for example a specific number of shakes of the sports ball 10 within a period of time. If that is the case, the spherical circuit board 20 may wake-up and be ready to record data. Otherwise, spherical circuit board 20 may go back to sleep and wait for another interrupt.

The 3D digital magnetic sensor 22A″ may be used to sense where the strongest magnetic force is coming from, generally used to detect magnetic north, but can also be used for measuring magnetic fields. In the present embodiment, the 3D digital magnetic sensor 22A″ may be a 3-axis sensor, which means it gives a 3D vector pointing to the strongest magnetic field. The 3D digital magnetic sensor 22A′ may be used to stabilize orientation calculations and also determine orientation with respect to the Earth. The 3D digital magnetic sensor 22A″ may include a 14-bit vector magnitude output. The three orthogonal magnetometer readings will be sampled and stored. Post-processing on the PC will be used to calculate spin rate and possibly other parameters. The sampling rate may be set as high as 6,400 Hz. However, the faster the sample rate the higher the power consumption. Thus, a sample rate of 100 Hz may be used to measure the spin rate of the sports ball 10 based on the sensed change in magnetic field direction.

A processor 24 and memory 26 are coupled to the spherical circuit board 20. The sensors 22 may be coupled to the processor 24 and memory 26 via a plurality of conductive pathways 28. The processor 24 may be used to control the various functions of the sports ball 10. The processor 24 may store a computer program or other programming instructions associated with the memory 26 to control the operation of sports ball 10. The processor 24 may comprise various computing elements, such as integrated circuits, microcontrollers, microprocessors, programmable logic devices, etc., alone or in combination to perform the operations described herein.

The memory 26 may be coupled to the processor 24 as well as other components of the sports ball 10. The memory 26 may be used to store various data monitored by the sensors 22 and utilized by the processor 24 and or other components of the sports ball 10. The memory 26 may include removable and non-removable memory elements such as RAM, ROM, flash, magnetic, optical, and/or other conventional memory elements. The above listing is given as an example and should not be seen in a limiting manner.

The memory 26 may be used to store programming data for instructing the processor 24 or other components of the sports ball 10 to perform certain steps. For example, the processor 24 may obtain the various readings from the plurality of sensors 22. Using the programming data, the processor 24 may calculate a variety of movement characteristics of the sports ball 10. For example, the processor 24 may calculate travel route, distance, time of flight, speed, trajectory height, spin rate, curve of the sports ball 10, release point as well as the barometric change in elevation with the respect in the relative change in movement of the sports ball 10 prior to the release point and to quantify the atmospheric air density due to pitcher's elevation above sea level and other characteristics.

The processor 24 may be coupled to a transmitting device 30. The transmitting device 30 may send all of the data calculated by the processor 24. In accordance with one embodiment, the transmitting device 30 may have an antenna. The antenna of the transmitting device 30 may be incorporated into and form part of the stitching 18. The data may be transmitted over any type of wireless network. For example, the wireless network may be though a 3G cellular communications, such as CDMA, EVDO, GSM/GPRS, or 4G cellular communications, such as WiMAX or LTE or the like. Alternatively, the wireless communication may by using a wireless local area network (WLAN), for example, using Wi-Fi or the like. The above is given as an example and should not be seen in a limiting manner. Other types of wireless networks may be used without departing from the spirit and scope of the present invention. For example, the wireless network may include any wireless communication network associated with a Personal Area Network (PAN), a Local Area Network (LAN), Metropolitan Area Network (MAN), or a Wide Area Network (WAN).

The transmitted data may be sent to a computing device for review. For example, the transmitted data may be sent to a desktop computer, a laptop, a tablet, smartphone, or other computing devices. In accordance with one embodiment, a dongle or similar device may be attached to the computing device. The dongle may be used to aid the computing device in the reception of the transmitted data. The transmitted data may be sent to a server for storage. This may allow a user to review the data at a later time via a computing device. In this embodiment, a user may have to login to a website or via a software application of a mobile computing device to review the transmitted data.

Alternatively, the sports ball 10 may have a data cable 44. In this embodiment, the data monitored by the sensors 22 may be stored in the memory 26. A user may then connect the sports ball 10 to a computing device via the data cable 44 to download the information. The use of the data cable 44.

The components on the spherical circuit board 20 may be powered by a battery 32. The battery 32 may be positioned on the exterior surface of the spherical circuit board 20. Alternatively, the battery 32 may be placed within the interior of the spherical circuit board 20. Placing the battery 32 within the interior of the spherical circuit board 20, may allow the spherical circuit board 20 to be easier to spin balance. In accordance with one embodiment of the present invention, the battery 32 is a spherical battery positioned within the spherical circuit board 20. In this embodiment, a cap 20A may be removed from the spherical circuit board 20. The battery 32 may then be placed within the interior of the spherical circuit board 20. The battery 32 may be a rechargeable battery. The battery 32 may be may charge using wired or wireless technology. By using wireless technology such as by using an electromagnetic field to transfers energy for recharging of the battery 32, the sports ball 10 may become easier to balance.

The spherical circuit board 20, sensors 22 and other associated components are mounted together to be properly balance. Computer programs may be used to mathematically calculate the location of each component on the spherical circuit board 20. If not properly balanced, the spherical circuit board 20 may have one or more counter weights 34. The counter weights 34 may be used to balance the spherical circuit board 20. When the spherical circuit board 20 is constructed, the spherical circuit board 20 may undergo a balance spin test. This may allow one to see if the spherical circuit board 20 is properly balanced. If not, counter weights 34 may be added in predetermined positions to balance the counter weights 34. A balance spin test may show the location and amount of counterweight needed to properly balance the spherical circuit board 20.

A mounting unit 36 may be used to position the spherical circuit board 20 within the core 12. The mounting unit 36 may be used to dynamically balance the spherical circuit board 20 while anchoring the spherical circuit board 20 within the core 12. In accordance with one embodiment, the mounting unit 36 may have a platform 38. The platform 38 may be a cylindrical ring 38A. The spherical circuit board 20 may be positioned within the cylindrical ring 38A. A plurality of mounting brackets 40 may be positioned around a perimeter of the cylindrical ring 38A. The mounting brackets 40 may be structural shaped brackets that are used to hold the spherical circuit board 20 in place and deflect kinematic energy.

To enhance the ability to measure, review and analyze the different motion characteristics of a pitched ball, the person throwing the sports ball 10 may have one or more wearable devices 46. The wearable devices may be placed on the wrist, shoe or other areas of the person. The wearable devices may monitor different movement characteristics of the person throwing the ball. The wearable device may monitor arm slot and movement, stride of the person throwing the sports ball 10 and other movement characteristics of the person throwing the ball. This information may be used in conjunction with the transmitted data from the sports ball 10 to enhance the ability to measure, review and analyze the different motion characteristics of a pitched ball.

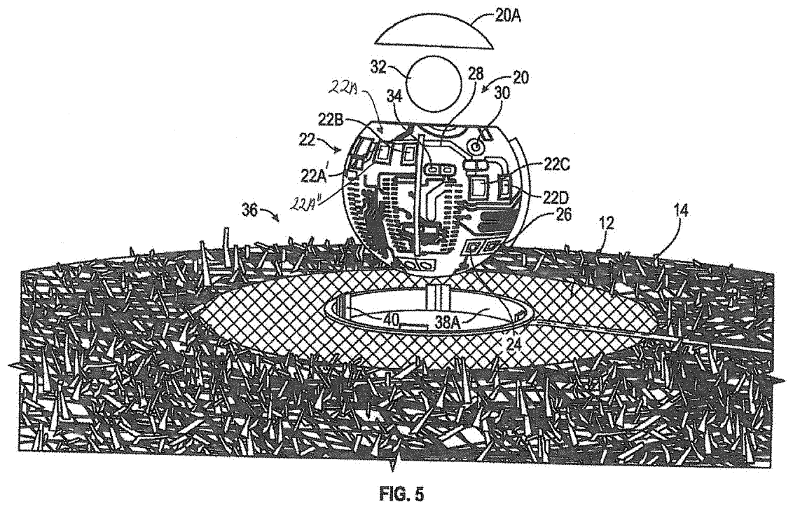

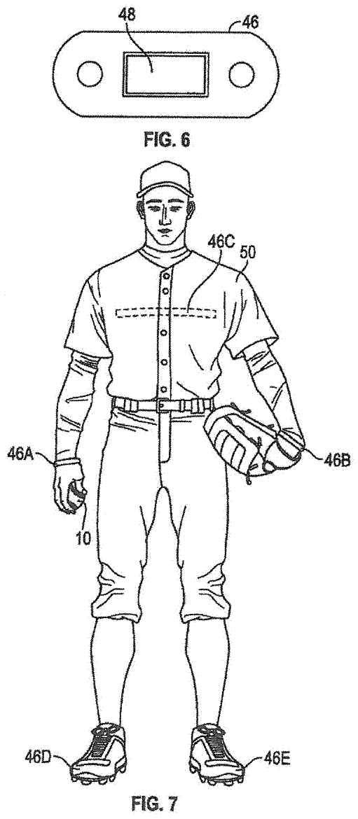

Referring to FIG. 6, one example of a wearable device 46 is shown. In this embodiment, the wearable device 46 is a foot pod that may be attached to the user's shoe. The foot pod may be used to measure the stride of the person throwing the sports ball 10 and other leg movement characteristics of the person throwing the sports ball 10. FIG. 5 shows just one example. As started above, wearable devices may be placed on the wrist or other areas of the person throwing the sports ball 10.

Referring to FIG. 7, an individual 50 is shown having a plurality of wearable devices 46 and the sports ball 10. In this embodiment, the individual 50 may have a wrist wearable device 46A located on a right wrist and another wrist wearable device 46B located on the left wrist. The wrist wearable devices 46A and 46B may be used to measure the movement of the arms of the individual 50 when throwing the sports ball 10. The wrist wearable devices 46A and 46B may be used to measure the grip strength. By monitoring the contraction and release of muscles located around the wrist, the approximate grip strength may be calculated. The above is given as an example and should not be seen in a limiting manner. The wrist wearable devices 46A and 46B may have other sensors that allow the wrist wearable devices 46A and 46B to monitor other characteristics than those mentioned above without departing from the spirit and scope of the present invention.

The wrist wearable devices 46A and 46B may monitor and record the arm movements, grip strength and other characteristics during the pitching process. One can review the recorded information to optimize the pitching performance of the individual 50. For example, the wrist wearable device 46B located on the left wrist may indicate that the position of the left wrist of the individual 50 may be either too close or too far away from the body and may be throwing the individual 50 slightly off balance. Similarly, the wrist wearable device 46A located on the right wrist may indicate that the individual 50 is not following through after the pitch. If the individual 50 is a right-handed pitcher and the wrist wearable device 46A monitors the grip strength, the wrist wearable device 46A may indicate a location of a release point of the pitch. By monitoring the location of the release point, the wrist wearable device 46A may indicate whether the release point of the individual 50 was too early or too late during the delivery.

The data monitored and recorded may be reviewed on a screen 48 (FIG. 5) of the wrist wearable devices 46A and 46B and/or transmitted to a computing device for analysis and review. For example, the transmitted data may be sent to a desktop computer, a laptop, a tablet, smartphone, or other computing devices. In accordance with one embodiment, a dongle or similar device may be attached to the computing device. The dongle may be used to aid the computing device in the reception of the transmitted data. The transmitted data may be sent to a server for storage. This may allow a user to review the data at a later time via a computing device. In this embodiment, a user may have to login to a website or via a software application of a mobile computing device to review the transmitted data.

In accordance with another embodiment, the wrist wearable devices 46A and 46B may have a port for downloading the data. The port may be a USB port or the like which may allow the wrist wearable devices 46A and 46B to be connected to a computer device to download the information recorded and stored.

The plurality of wearable devices 46 may include a chest strap device 46C. The chest strap device 46C may be used to monitor the movement of the chest, breathing patterns, heart rate as well as other characteristics of the individual 50 during the pitching process. One can review the recorded information to optimize the pitching performance of the individual 50. For example, the chest strap device 46C may indicate that the chest is rocking backwards during the wind-up or not in a proper location during the follow through throwing the individual 50 slightly off balance. The chest strap device 46C may indicate that the individual 50 is not breathing properly or that a heat rate of the individual 50 is too elevated and needs to calm down.

The data monitored and recorded may be reviewed on a screen 48 (FIG. 5) of the chest strap device 46C and/or transmitted to a computing device for analysis and review. For example, the transmitted data may be sent to a desktop computer, a laptop, a tablet, smartphone, or other computing devices. In accordance with one embodiment, a dongle or similar device may be attached to the computing device. The dongle may be used to aid the computing device in the reception of the transmitted data. The transmitted data may be sent to a server for storage. This may allow a user to review the data at a later time via a computing device. In this embodiment, a user may have to login to a website or via a software application of a mobile computing device to review the transmitted data.

In accordance with another embodiment, the chest strap device 46C may have a port for downloading the data. The port may be a USB port or the like which may allow the chest strap device 46C to be connected to a computer device to download the information recorded and stored.

The individual 50 may have a foot pod wearable device 46D located on a right shoe and another foot pod wearable device 46E located on the left shoe. The foot pod wearable devices 46D and 46E may be used to measure the movement of the feet of the individual 50 when throwing the sports ball 10 as well as other characteristics.

The foot pod wearable devices 46D and 46E may monitor and record the movement of the foot of the individual 50 as well as other characteristics during the pitching process. One can review the recorded information to optimize the pitching performance of the individual 50. For example, the foot pod wearable devices 46D and 46D may be used to indicate if the individual 50 is dragging the plant foot, not properly planning the plant foot, over striding with the lead foot, not striding wide enough with the lead foot, and the like.

The data monitored and recorded may be reviewed on a screen 48 (FIG. 5) of the foot pod wearable devices 46D and 46D and/or transmitted to a computing device for analysis and review. For example, the transmitted data may be sent to a desktop computer, a laptop, a tablet, smartphone, or other computing devices. In accordance with one embodiment, a dongle or similar device may be attached to the computing device. The dongle may be used to aid the computing device in the reception of the transmitted data. The transmitted data may be sent to a server for storage. This may allow a user to review the data at a later time via a computing device. In this embodiment, a user may have to login to a website or via a software application of a mobile computing device to review the transmitted data.

In accordance with another embodiment, the foot pod wearable devices 46D and 46D may have a port for downloading the data. The port may be a USB port or the like which may allow the chest strap device 46C to be connected to a computer device to download the information recorded and stored.

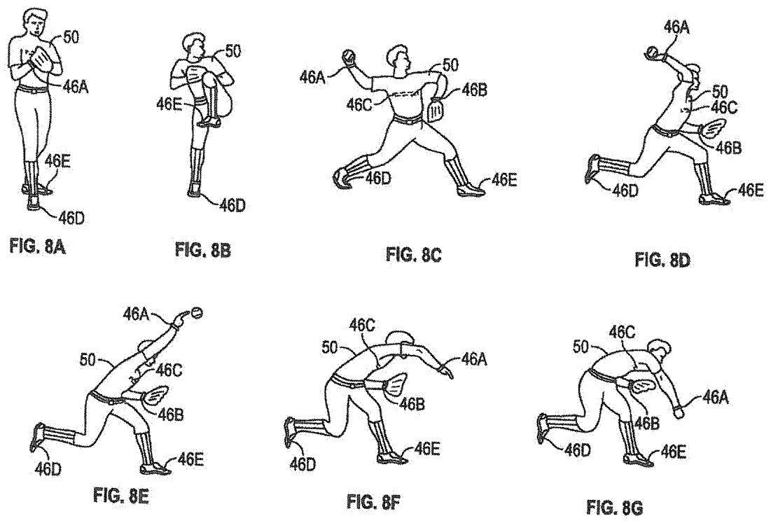

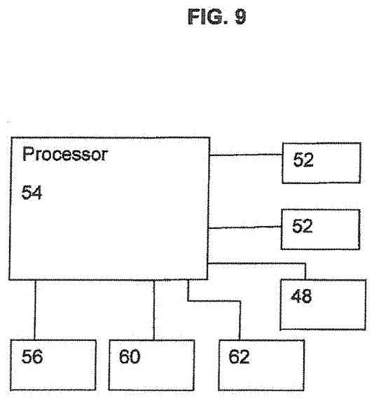

Referring to FIGS. 8A-80, the process of throwing the sports ball 10 may be shown with the individual 50 having the wrist wearable devices 46A and 46B, the chest strap device 46C and the foot pod wearable devices 46D and 46D. When throwing the sports ball 10, the wearable devices 46 may monitor the movement as well as other characteristics of the arm, feet, chest and other body parts of the individual 50. As may be seen, the pitching motion is a complex process requiring the proper movement of numerous elements of the body. By monitoring the different body parts, one may optimize the pitching performance of the individual 50.

Referring to FIG. 9, each of the wearable devices 46 may have one or more sensors 52. Each of the sensors 52 may be used to monitor different characteristics of the individual 10 to which the sensor is attached as described above. The sensors 52 may be coupled to a processor 54 and memory 56 via a plurality of conductive pathways. The processor 54 may comprise various computing elements, such as integrated circuits, microcontrollers, microprocessors, programmable logic devices, etc., alone or in combination to perform the operations described herein.

The memory 56 may be coupled to the processor 54 as well as other components of the wearable devices 46. The memory 56 may be used to store various data monitored by the sensors 52 and utilized by the processor 54 and or other components of the wearable devices 46. The memory 56 may include removable and non-removable memory elements such as RAM, ROM, flash, magnetic, optical, and/or other conventional memory elements. The above listing is given as an example and should not be seen in a limiting manner.

The memory 56 may be used to store programming data for instructing the processor 54 or other components of the wearable device 46 to perform certain steps. For example, the processor 54 may obtain the various readings from the plurality of sensors 52. Using the programming data, the processor 54 may calculate and analyze variety of movement characteristics of the individual 50 as well as other measured data.

The processor 54 may be coupled to a transmitting device 60. The transmitting device 60 may send all of the data calculated by the processor 54. In accordance with one embodiment, the transmitting device 60 may have an antenna. The data may be transmitted over any type of wireless network. For example, the wireless network may be though a 3G cellular communications, such as CDMA, EVDO, GSM/GPRS, or 4G cellular communications, such as WiMAX or LTE or the like. Alternatively, the wireless communication may by using a wireless local area network (WLAN), for example, using Wi-Fi or the like. The above is given as an example and should not be seen in a limiting manner. Other types of wireless networks may be used without departing from the spirit and scope of the present invention. For example, the wireless network may include any wireless communication network associated with a Personal Area Network (PAN), a Local Area Network (LAN), Metropolitan Area Network (MAN), or a Wide Area Network (WAN).

The transmitted data may be sent to a computing device for review. For example, the transmitted data may be sent to a desktop computer, a laptop, a tablet, smartphone, or other computing devices. In accordance with one embodiment, a dongle or similar device maybe attached to the computing device. The dongle may be used to aid the computing device in the reception of the transmitted data. The transmitted data may be sent to a server for storage. This may allow a user to review the data at a later time via a computing device. In this embodiment, a user may have to login to a website or via a software application of a mobile computing device to review the transmitted data.

Alternatively, the wearable devices 46 may have a data port 62. In this embodiment, the data monitored by the sensors 52 may be stored in the memory 56. A user may then connect the wearable devices 46 to a computing device via the data port 62 to download the information.

The wearable devices 46 may have a screen 48 coupled to the processor 54. The screen 48 may be used to display data analyzed by the processor 54.

The components in the wearable devices 46 may be powered by a battery. The battery may be a rechargeable battery. The battery may be may charge using wired or wireless technology.

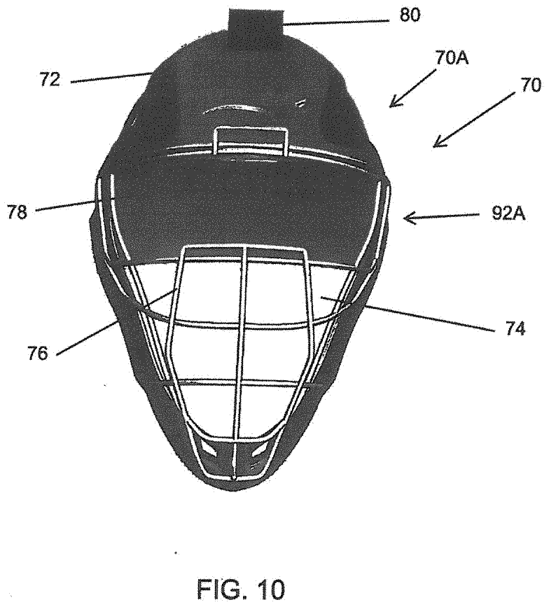

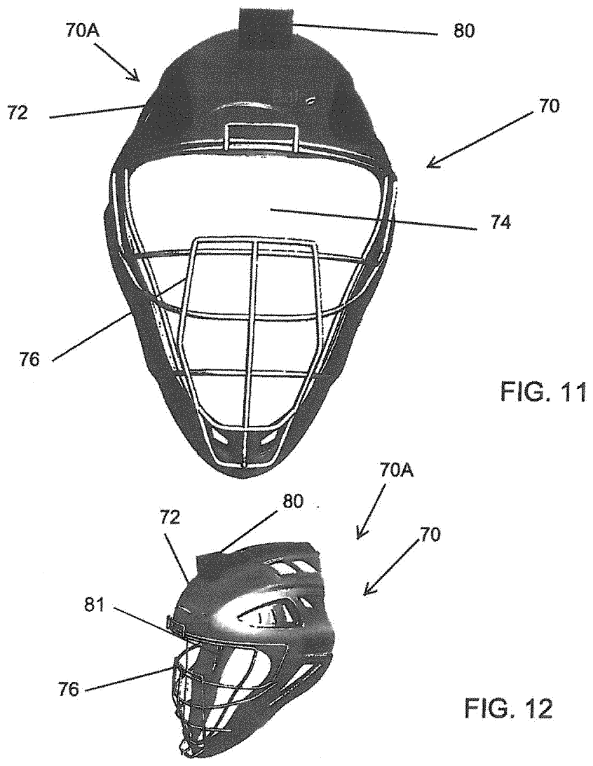

Referring to FIGS. 10-12, a wearable monitoring device 70 may be seen. In the embodiment shown, the wearable monitoring device 70 is a catcher's protective helmet 70A. However, the wearable monitoring device 70 may be other types of protective wear such as a batting helmet or the like. The above is given as an example and should not be seen in a limiting manner.

The wearable monitoring device 70 may have a catcher's half-hat helmet 72. The catcher's half-hat helmet 72 may have an opening 74 formed in a front area thereof. The opening 74 may allow a face of a user of the wearable monitoring device 70 to be partially exposed. A cage 76 may be attached to the catcher's half-hat helmet 72 covering the opening 74. The cage 76 may be designed to protect the face of the user exposed through the opening 74 while allowing the user to see and comfortably breath when wearing the catcher's half-hat helmet 72.

A monitoring device 80 may be attached to a top front surface of the catcher's half-hat helmet 72. The monitoring device 80 may be used to receive the data transmitted by the sports ball 10. The monitoring device 80 may further receive the data transmitted by the wearable devices 46. In accordance with one embodiment, the monitoring device 80 may also be used to record the movement of the individual 50 (FIG. 7-8G) throwing the sports ball 10. The monitoring device 80 may include a movement recording device such as a camera, 3D camera, video camera or similar devices that may record the movement of the individual 50. In accordance with one embodiment, the movement recording device may be a LIDAR (Light Detection and Ranging) device. A LIDAR device sends out pulse laser light signals which are used to illuminate the individual 50. The LIDAR device records the reflected pulses with a sensor. Differences in laser return times and wavelengths may then be used to make digital 3-D representations of the movement of the individual 50.

A communication device 81 may be coupled to an interior area of the catcher's half-hat helmet 72. The communication device 81 may allow a user of the wearable monitoring device 70 to wirelessly communicate with others such as the individual 50, coaches or other interested people. In accordance with one embodiment, the communication device 81 may use bone conduction technology.

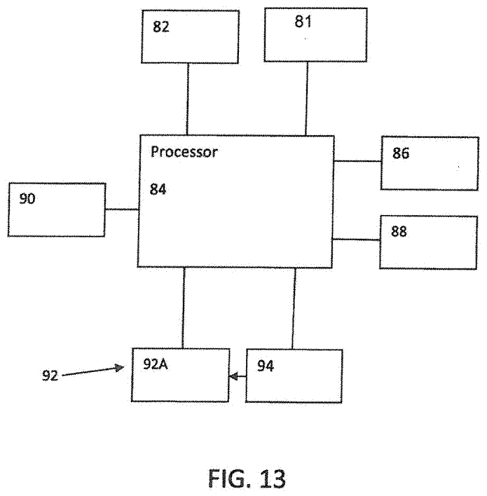

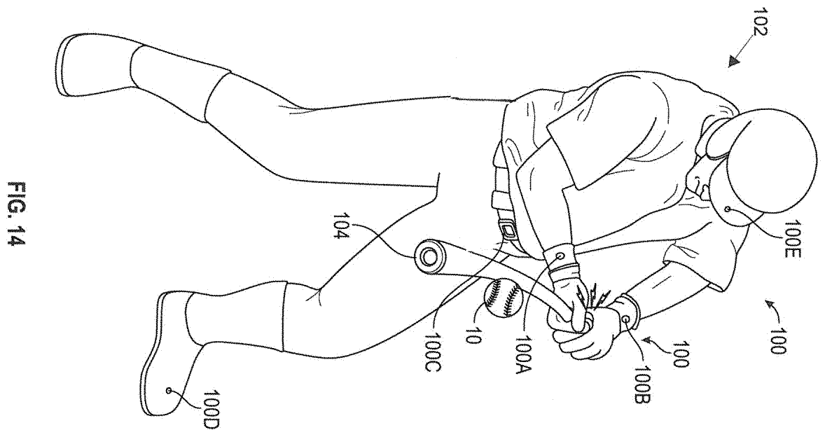

As may be seen in FIG. 13, the monitoring device 80 may have a receiver 82. The receiver 82 may be used to receive the data transmitted by the sports ball 10. The receiver 82 may also be used to receive the data transmitted by the wearable devices 46. The receiver 82 may be coupled to a processor 84 and memory 86 via a plurality of conductive pathways. The processor 84 may comprise various computing elements, such as integrated circuits, microcontrollers, microprocessors, programmable logic devices, etc., alone or in combination to perform the operations described herein.

The memory 86 may be coupled to the processor 84 as well as other components of the monitoring device 80. The memory 86 may be used to store various data received by the receiver 82. The memory 86 may store programming instructions which when executed by the processor 84 may analyze the data received by the receiver 82. For example, using the programming instructions, the processor 84 may calculate and analyze variety of movement characteristics of the sports ball 10, the individual 50 using the wearable devices 46, as well as other measured data. The memory 86 may include removable and non-removable memory elements such as RAM, ROM, flash, magnetic, optical, and/or other conventional memory elements. The above listing is given as an example and should not be seen in a limiting manner.

The processor 84 may be coupled to a transmitting device 88. The transmitting device 88 may send all of the data calculated by the processor 84. In accordance with one embodiment, the transmitting device 88 may have an antenna. The data may be transmitted over any type of wireless network. For example, the wireless network may be though a 3G cellular communications, such as CDMA, EVDO, GSM/GPRS, or 4G cellular communications, such as WiMAX or LTE or the like. Alternatively, the wireless communication may by using a wireless local area network (WLAN), for example, using Wi-Fi or the like. The above is given as an example and should not be seen in a limiting manner. Other types of wireless networks may be used without departing from the spirit and scope of the present invention. For example, the wireless network may include any wireless communication network associated with a Personal Area Network (PAN), a Local Area Network (LAN), Metropolitan Area Network (MAN), or a Wide Area Network (WAN).

A camera 90 may be coupled to the processor 84. The camera 90 may be like the embodiments disclosed above such as a camera, 3D camera, video camera, LIDAR or the like. The camera 90 may be used to take a video or monitor and record movement of the individual 50 throwing the sports ball 10. The data taken may be stored in the memory 86. Using the programming instructions, the processor 84 may calculate and analyze variety of movement characteristics of the individual 50 using the wearable devices 46 based on the data transmitted by the wearable devices 46 and the data recorded by the camera 90.

A display screen 92 may be coupled to the processor 84. The display screen 92 may be used to display the analyzed data calculated by the processor 84, the video captured by the camera 90 as well as other data. In accordance with one embodiment, the display screen 92 may be a head-up display 92A. In the embodiment having a head-up display 92A, the processor 84 may be coupled to picture generating unit 94. The processor 84 may transmit a signal to the picture generating unit 94 causing an image to be displayed on the head-up display 92A. The image may be the analyzed data calculated by the processor 84, the video captured by the camera 90 as well as other data. In accordance with one embodiment, the head-up display 92A may be a visor 78 coupled to a top section of the cage 76.

The wearable monitoring device 70 may be used by coaches trying to help train a pitcher. By viewing data displayed on the head-up display 92A or other display screen 92, the coach may provide immediate feedback to the individual 50. The wearable monitoring device 70 may also be used to help train umpires in calling balls and strikes. The above is given as examples and should not be seen in a limiting manner. The wearable monitoring device 70 may be used for other purposes then those disclosed above.

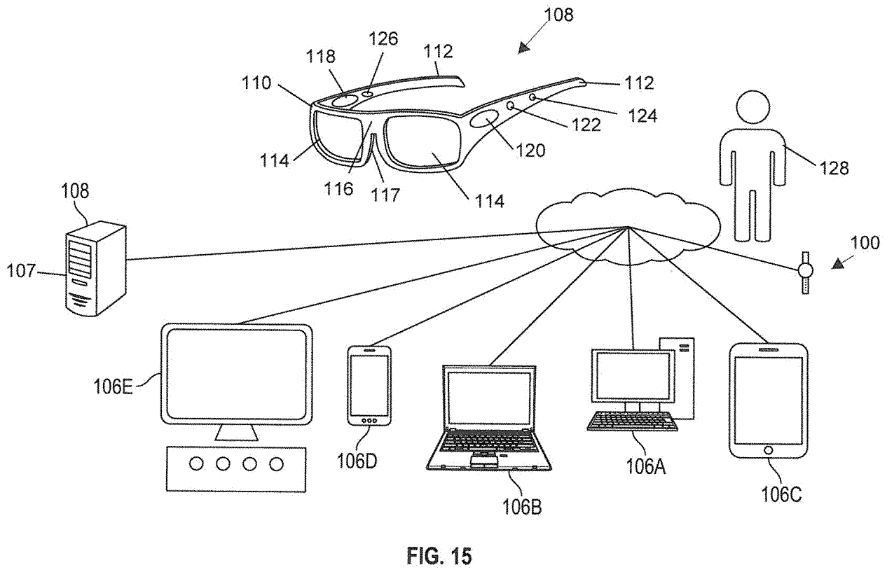

Referring to FIG. 14-15, the sports ball 10 may be used with wearable devices 100. The wearable device 100 may be similar to the wearable devices 46 being worn by the pitcher throwing the sports ball 10. In accordance with one embodiment, the wearable devices 100 may be worn by a batter 102 whom the sports ball 10 is being thrown towards by the pitcher. The wearable devices 100 may be placed on the wrist, waist, shoe or other areas of the batter. The wearable devices 100 may monitor different movement characteristics of the batter. The wearable device may monitor arm movement, stride of the batter hitting the sports ball 10, vibrations when contacting the sports ball with a bat 104 as well as other movement characteristics of the batter hitting the ball. This information may be used in conjunction with the transmitted data from the sports ball 10 to enhance the ability to measure, review and analyze the hitter's ability to hit the pitched sports ball 10.

In this embodiment, the batter 102 may have a wrist wearable device 100A located on a right wrist and another wrist wearable device 100B located on the left wrist. The wrist wearable devices 100A and 100B may be used to measure the movement of the arms of the batter 102 when swinging the bat 104 when trying to hit the sports ball 10. The wrist wearable devices 100A and 100B may be used to measure the grip strength, location, and/or vibration. The wrist wearable devices 100A and 100B may monitor the contraction and release of muscles located around the wrist, the approximate grip strength may be calculated. The wrist wearable devices 100A and 100B may use position sensors, motion sensors, GPS location or other means to monitor movement of the hands and to ensure that the hands of the batter 102 may be in the best set-up position for hitting. The wrist wearable devices 100A and 100B may use piezoelectric sensors or accelerometers to measure the vibrations. The vibrations recorded may indicated how solid the bat 104 hit the sports ball 10. The above is given as an example and should not be seen in a limiting manner. The wrist wearable devices 100A and 100B may have other sensors that allow the wrist wearable devices 100A and 100B to monitor other characteristics than those mentioned above without departing from the spirit and scope of the present invention.

The wearable devices 100 may include a waist wearable device 1000. The waist wearable device 100C may use position sensors, motion sensors, GPS location or other means to monitor movement of the waist of the batter 102. The waist wearable device 100C may be used to ensure the batter 102 is in a proper position and is rotating the waist to generate maximum power when hitting.

The wearable devices 100 may include a foot wearable device 100D. The foot wearable device 100D may use position sensors, motion sensors, GPS location or other means to monitor movement of the feet of the batter 102. The foot wearable device 100C may be used to ensure the batter 102 is in a proper position and is taking a proper stride to generate maximum power when hitting.

The wearable devices 100 may include a helmet wearable device 100E. The helmet wearable device 100E may use position sensors, motion sensors, GPS location or other means to monitor movement of the head of the batter 102. The helmet wearable device 100E may be used to ensure the head of the batter 102 is in a proper position to generate maximum power when hitting.

The data monitored and recorded by the wearable devices 100 may be transmitted wirelessly and/or wired to a computing device 106. The computing device 106 may be a desktop computer 106A, a laptop 106B, a tablet 106C, smartphone 106D, set-top box 106E or other computing type devices. In accordance with one embodiment, a dongle or similar device may be attached to the computing device 106. The dongle may be used to aid the computing device 106 in the reception of the transmitted data. In accordance with another embodiment, the wearable devices 100 may have a port for downloading the data.

In accordance with another embodiment, the wearable devices 100 may have a port for downloading the data. The port may be a USB port or the like which may allow the wearable devices 100 be connected to a computing device 106 to download the information recorded and stored.

The transmitted data may be sent to a server 107 for storage. This may allow a user to review the data at a later time via a computing device 106. In this embodiment, a user may have to login to a website or via a software application of a mobile computing device to review the transmitted data.

As may be seen in FIG. 15, a pair of smart glasses 108 may be provided. The smart glasses 108 may have a frame 110. The frame 110 may have a pair of temples 112. The temples may be used to secure the frame 110 over a pair of ears of a user. The frame 110 may have a pair of lenses 114. The frame 110 may be connected across the lenses 114 by a bridge 116. A nose pad 117 may be provided to support the frame 110 on a nose of the user.

In accordance with one embodiment, the lenses 114 may be an optical head-mounted display (OHMD), a computerized Internet connected glasses with transparent heads-up display (HUD), or an augmented reality overlay that has the capability of reflecting projected digital images.

The smart glasses 108 may have a transmitter/receiver 118 located thereon. The transmitter/receiver 118 maybe used to allow the smart glasses 108 to send and receive data. For example, the transmitter/receiver 118 may be used to receive the data transmitted by the sports ball 10. The monitoring device 80 may further receive the data transmitted by the wearable devices 46 and/or the data transmitted by the wearable devices 100. The transmitter/receiver 118 may be used to transmit data from the smart glasses 108 as disclosed below.

The transmitter/receiver 118 may be coupled to a processor 122 and memory 124 via a plurality of conductive pathways formed in the frame 110 of the smart glasses 108. The processor 122 may comprise various computing elements, such as integrated circuits, microcontrollers, microprocessors, programmable logic devices, etc., alone or in combination to perform the operations described herein.

The memory 124 may be coupled to the processor 122 as well as other components of the smart glasses 108. The memory 124 may be used to store various data to transmit or received by the transmitter/receiver 118. The memory 124 may store programming instructions which when executed by the processor 122 may analyze the data received by the transmitter/receiver 118. For example, using the programming instructions, the processor 122 may calculate and analyze variety of movement characteristics of the sports ball 10, the individual 50 using the wearable devices 46, the batter 102 using wearable 100 as well as other measured data. The memory may include removable and non-removable memory elements such as RAM, ROM, flash, magnetic, optical, and/or other conventional memory elements. The above listing is given as an example and should not be seen in a limiting manner.

The processor 122 may be coupled to a transmitter/receiver 118 via a plurality of conductive pathways formed in the frame 110 of the smart glasses 108. The transmitter/receiver 118 may be used to transmit and receive data. The data may be transmitted/received over any type of wireless network 124. For example, the wireless network may be though a 3G cellular communications, such as CDMA, EVDO, GSM/GPRS, or 4G cellular communications, such as WiMAX or LTE, 5G or the like. Alternatively, the wireless communication may by using a wireless local area network (WLAN), for example, using Wi-Fi or the like. The above is given as an example and should not be seen in a limiting manner. Other types of wireless networks may be used without departing from the spirit and scope of the present invention. For example, the wireless network may include any wireless communication network associated with a Personal Area Network (PAN), a Local Area Network (LAN), Metropolitan Area Network (MAN), or a Wide Area Network (WAN).

The smart glasses 108 may have a dual-output acoustic transducer module 126. The dual-output acoustic transducer module 122 may be attached to one or both of the pair of temples 112 of the smart glasses 108. The dual-output acoustic transducer module 126 may be coupled to the processor 122 via a plurality of conductive pathways formed in the frame 110 of the smart glasses 108. The dual-output acoustic transducer module 126 may be used to allow sound waves to be transmitted to the ear drum, or use bone conductive transmission to transmit the sound waves through bones in the user's skull.

The smart glasses 108 may be worn by the batter 102, a coach or a third-party person 128. Multiple smart glasses 108 may be used wherein one is provided to the batter 102, a coach and multiple third-party people 128. By receiving the data transmitted by the sports ball 10, the data transmitted by the wearable devices 46 and/or the data transmitted by the wearable devices 100, the batter 102 may use the transmitted data to learn how to read pitches and/or correct body movement to improve his/her hitting ability. Coaches wearing the smart glasses 108 may use the data to help train the batter 102 and/or the pitcher. The above is given as examples and should not be seen in a limiting manner.

The smart glasses 108 may also be used by the third-party person 128. This may allow the third-party person 128 to view the data in real time as well as to log into the server 107 to review past data and/or stored data from other players. Based on reviewing the statistical data stored on the server 107 and/or in-person real time data, the third-party person 128 may want to make a sports wager. The server 107 may host and/or allow access to a sports wagering website. The sports wagering website may allow the third-party person 128 to review the past data and/or stored data from players and place wagers on different sporting events through the smart glasses 108. Different betting options may be displayed on one or both of the lenses 114. The third-party person 122 may use different input/output devices 120 of the smart glasses 108 to select and enter a desired betting option that may be displayed on one of the lenses 114. Alternatively, the third-party person 128 after reviewing the real time data and/or data stored on the server 107 may use the smart glasses 108 to access a sports wagering website unaffiliated with the server 107 to make a wager in a similar fashion as disclosed above. The third-party person 128 may also go to a sports book room to make an in-person wager based on the data reviewed on the smart glasses 108.

The foregoing description is illustrative of particular embodiments of the application, but is not meant to be a limitation upon the practice thereof. The following claims, including all equivalents thereof, are intended to define the scope of the application.

What is claimed is:

1. A sports ball system for calculating movement characteristics comprising:
a sports ball monitoring movement characteristics of the sports ball;
a plurality of wearable devices worn by a batter whom the sports ball is being thrown towards, wherein at least one of the plurality of wearable devices receiving and displaying data transmitted by the sports ball and a remaining plurality of wearable devices recording data relating to movement of the batter.

2. The sports ball system in accordance with claim 1, the at least one of the plurality of wearable devices receiving and displaying data transmitted by the sports ball is a pair of smart glasses.

3. The sports ball system in accordance with claim 1, wherein one of the remaining plurality of wearable devices comprises at least one wrist sensor.

4. The sports ball system in accordance with claim 1, wherein one of the remaining plurality of wearable devices comprises at least one foot sensor.

5. The sports ball system in accordance with claim 1, wherein one of the remaining plurality of wearable devices comprises at least one waist sensor.

6. The sports ball system in accordance with claim 1, wherein one of the remaining plurality of wearable devices comprises at least one batting helmet sensor.

7. The sports ball system in accordance with claim 1, the pair of smart glasses is worn by the batter.

8. The sports ball system in accordance with claim 1, the pair of smart glasses is worn by a third party.

9. The sports ball system in accordance with claim 8, wherein the pair of smart glasses worn by the third party allowing access to a sports betting website.

10. The sports ball system in accordance with claim 1, wherein the sports ball comprises:
a spherical core;
a spherical circuit board mounted within the spherical core monitoring and calculating the movement characteristics of the sports ball, wherein the spherical circuit board comprises:
a controller;
an accelerometer; and
a magnetometer;
a mounting unit formed within the spherical core for securing the spherical circuit board within the spherical core, the mounting unit comprising:
a platform housed within the spherical core; and
a plurality of mounting brackets positioned around a perimeter of the platform, the mounting brackets used to hold the spherical circuit board in place and deflect kinematic energy;
winding wrapped around the spherical core; and
a cover positioned around the winding.

11. The sports ball in accordance with claim 10, wherein the sports ball comprises counterweights positioned on the spherical circuit board.

12. A sports ball system for calculating movement characteristics comprising:
a sports ball recording and transmitting movement characteristics of the sports ball;
a plurality of wearable devices worn by a batter whom the sports ball is being thrown towards, at least one of the plurality of wearable devices receiving and displaying data transmitted by the sports ball and a remaining plurality of wearable devices recording data relating to movement of the batter, wherein the at least one of the plurality of wearable devices receiving and displaying data transmitted by the sports ball is a pair of smart glasses worn by the batter.

13. The sports ball system in accordance with claim 12, wherein one of the remaining plurality of wearable devices comprises at least one wrist sensor.

14. The sports ball system in accordance with claim 12, wherein one of the remaining plurality of wearable devices comprises at least one foot sensor.

15. The sports ball system in accordance with claim 12, wherein one of the remaining plurality of wearable devices comprises at least one waist sensor.

16. The sports ball system in accordance with claim 12, wherein one of the remaining plurality of wearable devices comprises at least one batting helmet sensor.

17. The sports ball system in accordance with claim 12, comprising a second pair of smart glasses worn by a third party, the smart glasses allowing access to a sports betting website.

18. The sports ball system in accordance with claim 21, wherein the sports ball comprises:
a spherical core;
a spherical circuit board mounted within the spherical core monitoring and calculating the movement characteristics of the sports ball, wherein the spherical circuit board comprises:
a controller;
an accelerometer, and
a magnetometer;
a mounting unit formed within the spherical core for securing the spherical circuit board within the spherical core, the mounting unit comprising:
a platform housed within the spherical core; and
a plurality of mounting brackets positioned around a perimeter of the platform, the mounting brackets used to hold the spherical circuit board in place and deflect kinematic energy;
winding wrapped around the spherical core; and
a cover positioned around the winding.

19. A sports ball system for calculating movement characteristics comprising:
a sports ball recording and transmitting movement characteristics of the sports ball;
a plurality of wearable devices worn by a batter whom the sports ball is being thrown towards, wherein at least one of the plurality of wearable devices is a pair of smart glasses worn by the batter receiving and displaying data transmitted by the sports ball and a remaining plurality of wearable devices recording data relating to movement of the batter, wherein the remaining plurality of wearable devices comprises:
at least one wrist sensor;
at least one foot sensor;
at least one waist sensor; and
at least one batting helmet sensor.

20. The sports ball system in accordance with claim 19, comprising a second pair of smart glasses worn by a third party, the smart glasses allowing access to a sports betting website.
